(19) United States
(12) Patent Application Publication
(10) Pub. No.: US 2004/0241108 A1
(43) Pub. Date: Dec. 2, 2004

(54) Oral compositions

(76) Inventors: Peter William Stanier, Sandbach (GB); Simon Stebbing, Warrington (GB)

Correspondence Address: PILLSBURY WINTHROP, LLP, P.O. BOX 10500, MCLEAN, VA, 22102, US

(21) Appl. No.: 10/490,486

(22) PCT Filed: Oct. 1, 2002
(86) PCT No.: PCT/GB02/04424

(30) Foreign Application Priority Data
Nov. 1, 2001 (GB) 0126244.3

(51) Int. Cl. A61K 7/16

(52) U.S. Cl. 424/49

(57) ABSTRACT

An oral composition comprises a particulate amorphous silica and a cationic antibacterial agent characterised in that the particles of said amorphous silica have a polyether glycol deposited thereon. The silica used in the composition of the invention has a good compatibility with the cationic antibacterial agent.

This invention relates to oral compositions containing silica and in particular to oral compositions containing a modified silica and a cationic antibacterial agent.

The use of antibacterial agents, including cationic antibacterial agents, in oral hygiene compositions has been widely advocated as a means of reducing the oral bacterial plaque population and this may be beneficial in the treatment of periodontal disease, calculus, and/or caries.

Whilst mouthwashes comprising cationic antibacterial agents are available, these suffer the disadvantage that the cationic antibacterial agents tend to leave a brown stain, due to interaction of the agent with plaque. Such a drawback may, in principle, be minimised by using the antibacterial agent in a dentifrice, so that the abrasive included therein may remove the plaque. In practice, however, there are found to be severe problems in providing a satisfactory formulation, because cationic antibacterial agents are intrinsically incompatible with many of the other conventional elements of a dentifrice formulation, and this incompatibility drastically reduces the biological activity of the cationic agent. In addition, the cationic antibacterial agents are recognised as having a bitter taste, which needs to be masked to provide a product which is acceptable to the consumer.

EP-A-0 364 245 (to Beecham Group plc) discloses dentifrices comprising a bis-biguanide antibacterial agent such as chlorhexidine in combination with a non-ionic surfactant, a non-ionic thickening agent and an abrasive such as a silica with a low anion content, selected for compatibility with the antibacterial agent. In addition, EP-A-0 368 130 (to Procter & Gamble Co.) generically discloses dentifrices comprising a cationic antibacterial agent in combination with a non-ionic surfactant, a non-ionic thickening agent, a non-ionic humectant and a silica abrasive having good compatibility with cationic antibacterial agents. The specific examples provided therein are of a chlorhexidine-containing dentifrice in which the compatible silica is a special experimental grade provided by J.M. Huber Corporation and characterised by, inter alla, a low sulphate ion content (less than 0.25%), a BET surface area of about 10 to 300 m²g⁻¹ and the presence of from 10 to 300 parts per million of alkaline earth metal ions. These ions are introduced during the final stage of preparation of the silica, to produce a special surface-coated silica.

EP-A-0 315 503 (to Rhone-Poulenc Chimie) discloses the suitability of certain grades of silicas for use in chlorhexidine-containing dentifrices, which silicas are characterised by, inter alla, a low anion content (less than 1%).

Silica has found widespread use in oral compositions in which it can be used as a thickening agent, an abrasive or cleaning agent, or as a sensory mouthfeel agent. An abrasive/cleaning silica may also provide some thickening, especially when deliberately produced to have bifunctional properties. Cationic antibacterial agents are known to interact with silica in oral compositions and it is expected that this interaction will reduce the effectiveness of the antibacterial agent.

The use of a silica, the particles of which have been treated with a polyether glycol, in an oral composition has been disclosed in PCT application WO 99/63958 but the use in oral compositions containing cationic antibacterial agents is not disclosed therein.

It has now been surprisingly discovered that treatment of the surface of a silica with certain polyether glycols can reduce the interaction of the silica with cationic antibacterial agents.

According to the invention an oral composition comprises a particulate amorphous silica and a cationic antibacterial agent characterised in that the particles of said amorphous silica have a polyether glycol deposited thereon.

The presence of the polyether glycol deposited upon the particles of silica has been shown to markedly increase the compatibility of the silica with cationic antibacterial agents and, in particular, with guanidine compounds, especially chlorhexidine, as demonstrated by the compatibility test defined hereinafter.

Silica has been used in oral compositions principally to provide a thickening effect or to act as an abrasive agent. Some silicas, the so-called bifunctional silicas, can provide both these functionalities. More recently, special silicas have been incorporated into dentifrices to provide novel sensory mouthfeel or visual effects. Treatment of any of these types of silica with a polyether glycol has been shown to improve the compatibility of the silica with cationic antibacterial agents.

Therefore, the amorphous silica used as a base upon which to deposit the polyether glycol (“naked silica”) may be any silica conventionally used in oral compositions. Preferably, the naked silica has a CTAB (hexadecyltrimethyl ammonium bromide) surface area in the range 5 m²g⁻¹ to 400 m²g⁻¹. More preferably, the CTAB surface area is in the range 10 m²g⁻¹ to 250 m²g⁻¹. Most preferably, the CTAB surface area is in the range 10 m²g⁻¹ to 100 m²g⁻¹.

The oil absorption of the naked silica is preferably in the range 40 to 400 cm³/100 g. When the silica is a thickening silica the oil absorption is more preferably in the range 200 to 400 cm³/100 g. An abrasive silica more preferably has an oil absorption in the range 40 to 140 cm³/100 g and a bifunctional silica has a more preferred oil absorption in the range 120 to 250 cm³/100 g.

The particles of silica generally have a weight mean particle size in the range 3 to 20 μm, as determined using a Malvern Mastersizer®. Preferably, the weight mean particle size of the silica is in the range 3 to 15 μm using a Malvern Mastersizer®. The silica may also be in the form of sensory particles, which are agglomerates or aggregates of silica particles, particularly an agglomerate that breaks down readily when the oral composition is used. Generally, the aggregates of silica particles do not break down when the oral composition is used. The agglomerated silica is preferably composed of silica particles having a weight mean particle size as mentioned hereinbefore and the agglomerates or aggregates preferably have a weight mean particle size in the range 50 to 1000 μm, as determined by sieving. More preferably, the weight mean particle size of sensory particles, as determined by sieving, is in the range 100 to 700 μm, most preferably 100 to 500 μm. When the silica is in the form of an agglomerate, the polyether glycol may be deposited onto the silica particles before or after the particles are formed into the agglomerated material.

The naked silica preferably has a pH value in the range 3 to 9, more preferably in the range 5 to 8.

The amount of water present on the naked silica, as measured by the ignition loss at 1000° C. is preferably up to 30 percent by weight and more preferably up to 15 percent by weight. Usually the ignition loss at 1000° C. is more than 4 percent by weight.

An objective of the invention is to provide an oral composition containing a cationic antibacterial agent wherein the effect of silica present in the composition on the antibacterial activity of the composition is minimised. Consequently, the treated silica preferably has a compatibility with a cationic antibacterial agent of at least 50 percent, as measured by the compatibility test defined hereinafter. In this test, chlorhexidine digluconate is used as the cationic antibacterial agent. It is believed that a compatibility with chlorhexidine digluconate is indicative of a compatibility with cationic antibacterial agents in general. Preferably, the compatibility is at least 60 percent and most preferably at least 70 percent according to this test. A preferred silica is also a treated silica which has an improved compatibility with cationic antibacterial agents, as measured by this test, compared to the naked silica from which the treated silica is prepared. Preferably, the treated silica has a compatibility with cationic antibacterial agents of at least 30 percentage units higher than the compatibility of the naked silica from which it was produced, both compatibilities being measured by the above-mentioned test.

The amount of silica present in the oral composition depends upon the function it performs in the composition. Usually, the amount is in the range 0.1 to 35 percent by weight of the oral composition. When it is a thickening silica, it is preferably present in the range 1 to 15 percent by weight, when it is an abrasive silica it is preferably present in the range 4 to 35 percent by weight and when it is a sensory particle it is preferably present in the range 0.1 to 10 percent by weight.

The polyether glycol used to deposit onto the silica can be any polyether glycol. Particularly useful are the polyalkylene glycols such as polyethylene glycols and polypropylene glycols.

The amount of polyether glycol deposited on the silica can vary widely and depends, to some extent, on the nature of the silica, the purpose for which the silica is present in the oral composition and the nature of the polyether glycol. Usually, the amount of polyether glycol is up to 30 percent by weight based on the weight of naked silica. Preferably, the amount of polyether glycol is up to 15 percent and frequently the amount is less than 5 percent by weight with respect to naked silica. Normally, the amount of polyether glycol present on the silica is greater than 0.1 percent by weight based on weight of naked silica and more commonly more than 0.5 percent by weight based on weight of naked silica is used.

The molecular weight of useful polyether glycols depends upon the polyether glycol used. When polyethylene glycol is used, the average molecular weight is preferably between 200 and 20,000.

Suitable cationic antibacterial agents for use in oral compositions of the invention include, for example:

(i) quaternary ammonium compounds, such as those in which one or two of the substituents on the quaternary nitrogen has from 8 to 20, preferably from 10 to 18 carbon atoms and is preferably an alkyl group, which may optionally be interrupted by an amide, ester, oxygen, sulphur, or heterocyclic ring, whilst the remaining substituents have a lower number of carbon atoms, for instance from 1 to 7, and are preferably alkyl, for instance methyl or ethyl, or benzyl. Examples of such compounds include benzalkonium chloride, dodecyl trimethyl ammonium chloride, benzyl dimethyl stearyl ammonium chloride, hexadecyltrimethyl ammonium bromide, benzethonium chloride (diisobutyl phenoxyethoxyethyl dimethyl benzyl ammonium chloride) and methyl benzethonium chloride;

(ii) pyridinium and isoquinolinium compounds, including hexadecylpyridinium chloride and alkyl isoquinolinium bromides;

(iii) pyrimidine derivatives such as hexetidine (5-amino-1,3-bis(2-ethylhexyl)-5-methyl-hexahydropyrimidine);

(iv) amidine derivatives such as hexamidine isethionate (4,4′-diamidino-α,ω-diphenoxy-hexane isethionate);

(v) bispyridine derivatives such as octenidine dihydrochloride (N,N′[1,10-decanediyldi-1(4H)-pyridinyl4-ylidene]-bis(1-octanamine) dihydrochloride); and

(vi) guanides, for example, mono-biguanides such as p-chlorobenzyl-biguanide and N′-(4-chlorobenzyl)-N″-(2,4-dichlorobenzyl)biguanide, poly(biguanides) such as polyhexamethylene biguanide hydrochloride, and bis-biguanides of the general formula (1):

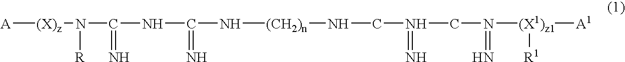

in which A and A¹ each represent (i) a phenyl group optionally substituted by (C₁₋₄) alkyl, (C₁₋₄) alkoxy, nitro, or halogen, (ii) a (C₁₋₁₂) alkyl group, or (iii) a (C₄₋₁₂) alicyclic group; X and X¹ each represent (C₁₋₃) alkylene; R and R¹ each represent hydrogen, (C₁₋₁₂) alkyl, or aryl(C₁₋₆) alkyl; Z and Z1 are each 0 or 1; n is an integer from 2 to 12; and the polymethylene chain (CH₂)ₙ may optionally be interrupted by oxygen or sulphur or an aromatic (for instance phenyl or naphthyl) nucleus; and orally acceptable acid addition salts thereof; examples of such bis-biguanides include chlorhexidine and alexidine. Suitable acid addition salts of the bis-biguanides of general formula (1) include the diacetate, the dihydrochloride and the digluconate. Suitable acid addition salts of chlorhexidine are those which have a water solubility at 20° C. of at least 0.005% w/v and include the digluconate, diformate, diacetate, dipropionate, dihydrochloride, dihydroiodide, dilactate, dinitrate, sulphate, and tartrate salts. Preferably the salt is the dihydrochloride, diacetate or digluconate salt of chlorhexidine. Suitable acid addition salts of alexidine include the dihydrofluoride and the dihydrochloride salts.

Suitably, the cationic antibacterial agent is selected from benzethonium chloride, octenidine, hexetidine, hexamidine, cetyl pyridinium chloride, chlorhexidine or alexidine. Advantageously, the cationic antibacterial agent is present in the range 0.005 to 10 percent, preferably 0.005 to 5 percent, more preferably 0.005 to 2.5 percent by weight of the oral composition.

Generally, in addition to the treated silicas and a cationic antibacterial agent, the oral composition will contain water and a humectant.

Usually the oral composition will be in the form of a toothpaste, gel, cream or liquid, of the opaque, translucent or transparent variety. The exact physical properties of the oral composition may be controlled for example by suitable adjustment of the quantities and nature of the water, humectant and thickener, which may be a thickening silica with a polyether glycol deposited thereon as hereinbefore described.

The humectant component of such a composition may comprise a polyol such as glycerol, sorbitol syrup, polyethylene glycol, polypropylene glycol, lactitol, xylitol or hydrogenated corn syrup. The total amount of humectant may, for example, be in the range of 10 to 85 percent by weight of the composition.

The water content of such a composition typically ranges from 1 to about 90 percent by weight, preferably from about 10 to about 60 percent by weight, more preferably from about 15 to about 50 percent by weight. In the case of transparent pastes, a preferred range is from about 1 to about 35 percent by weight

The oral composition of the invention frequently comprises one or more additional components, such as those described below.

The composition of the invention may include one or more surfactants, preferably selected from non-ionic, cationic and amphoteric surfactants, and mixtures thereof, all being suitable for oral use. The amount of surfactant present in the composition of the invention is typically from about 0.005 to about 20 percent by weight, preferably 0.1 to 10 percent, more preferably 0.1 to 5 percent by weight of the oral composition (based upon 100 percent activity of the surfactant).

Suitable non-ionic surfactants include, for example, polyethoxylated sorbitol esters, in particular polyethoxylated sorbitol monoesters; polycondensates of ethylene oxide and propylene oxide (poloxamers); condensates of propylene glycol; polyethoxylated hydrogenated castor oil and sorbitan fatty esters.

Suitable cationic surfactants include the D, L-2-pyrrolidone-5-carboxylic acid salt of ethyl-N-cocoyl-L-arginate.

Suitable amphoteric surfactants include, for example, long chain imidazoline derivatives; long chain alkyl betaines and long chain alkyl amidoalkyl betaines such as cocamidopropyl betaine and sulphobetaines.

The oral composition of the invention may also incorporate suitable well-known polymer suspending or thickening agents. Suitable thickening agents include, for example, sodium carboxymethyl cellulose, (C₁₋₆) alkylcellulose ethers, for instance methylcellulose, hydroxy-(C₁₋₆) alkylcellulose ethers, for instance hydroxypropylcellulose, (C₂₋₆) alkylene oxide modified (C₁₋₆) alkylcellulose ethers, for instance hydroxypropyl methylcellulose, and mixtures thereof. Other natural or synthetic gums and polymers such as gum tragacanth, polyvinylpyrrolidone, starch and polyacrylates such as Carbapol™ polymers can be used. These agents (which may be used singly or as mixtures of two or more of the above materials) may be present in the composition in a total amount of from about 0.01 to about 30 percent by weight, preferably 0.1 to 5 percent by weight of the oral composition.

The oral composition may further comprise an ionic fluorine-containing compound characterised by its ability to release fluoride ions in water and by substantial freedom from reaction with other compounds of the oral composition. This can include ionic fluorides and ionic monofluorophosphates which may be incorporated into the formulation to provide between 100 and 3000 ppm, preferably 500 to 2000 ppm of fluoride in the formulation. Preferably, the ionic fluoride or monofluorophosphate is an alkali metal fluoride or monofluorophosphate, for instance sodium fluoride or sodium monofluorophosphate, respectively.

One or more other components that are conventionally found in an oral composition may be present in the oral composition, providing that they do not interact with the cationic antibacterial agent in any appreciable way. These include the following; flavouring substances such as peppermint, spearmint and aniseed; artificial sweeteners; perfume or breath freshening substances; antistain additives, for example a peroxydiphosphate salt such as tetrapotassium peroxydiphosphate; pearlescing agents; opacifiers; pigments and colourings; preservatives; other therapeutic agents including anti-caries, anti-plaque, anti-tartar agents and anti-hypersensitivity agents; proteins; enzymes; salts; baking soda and pH adjusting agents.

Oral compositions in accordance with the invention may be made by conventional methods for preparing such compositions. Pastes and creams may be prepared by conventional techniques, for example using high shear mixing systems under vacuum.

The polyether glycol may be deposited on the silica in any suitable manner. When the polymer is a polyalkylene glycol, it is convenient to combine the treatment with the conventional preparation of a silica suitable for use in an oral composition. For example, during the preparation of a precipitated silica, an aqueous slurry of the silica is formed. It is convenient to add polyalkylene glycol to this slurry and mix for a period, typically from 5 to 60 minutes, at a temperature in the range 20 to 95° C. and at a pH of 2 to 7. In a preferred embodiment, the polyalkylene glycol is added to a precipitated silica slurry after neutralisation has been completed at a pH of 4 to 5, and a temperature of 60 to 70° C. for a period of about 10 to about 30 minutes, prior to the filtration/washing step conventionally used in such processes. The slurry of treated silica is then usually filtered and washed to remove residual electrolyte, frequently to below 2 percent electrolyte by weight, based on the dry weight of silica. After washing, the slurry is filtered and the filter cake is dried, typically by flash drying to remove the water rapidly from the silica so that the inherent structure is maintained, and comminuted to an appropriate particle size.

An alternative route for application of a polyalkylene glycol to the silica particles is to take dry particulate silica, slurry it in water and add the polyalkylene glycol to the slurry until the polymer is fully dispersed in the slurry. The treated particles thus obtained are then filtered, dried and (optionally) comminuted to the required particle size.

A further alternative treatment method is to spray a solution of a polyalkylene glycol onto silica particles as a coating, for example in a fluidised bed, followed by a drying and (optionally) a comminution step. All of these methods are effective in applying a polyalkylene glycol to the particulate material, although application during the silica manufacturing process is preferred on cost and ease of processing grounds.

The silicas used in this invention are characterised by the following test methods.

CTAB Surface Area

The CTAB surface area is determined using the technique of ASTM D3765 using CTAB at pH 9 and taking 0.35 nm² as the projected area of the CTAB molecule.

Oil Absorption

The oil absorption is determined by the ASTM spatula rub-out method (American Society of Test Material Standards D 281). The test is based on the principle of mixing linseed oil with the silica by rubbing with a spatula on a smooth surface until a stiff putty-like paste is formed which will not break or separate when it is cut with a spatula. The oil absorption is then calculated from the volume of oil (V cm³) used to achieve this condition and the weight, W, in grams, of silica by means of the equation:

Oil absorption=(V×100)/W, i.e. expressed in terms of cm³ oil/100 g silica.

Weight Mean Particle Size by Malvern Mastersizer®

The weight mean particle size of the silica is determined using a Malvern Mastersizer® model S, with a 300 RF lens and MS17 sample presentation unit. This instrument, made by Malvern Instruments, Malvern, Worcestershire uses the principle of Fraunhofer diffraction, utilising a low power He/Ne laser. Before measurement the sample is dispersed ultrasonically in water for 5 minutes to form an aqueous suspension. The Malvern Mastersizer® measures the weight particle size distribution of the silica. The weight mean particle size (d₅₀) or 50 percentile is easily obtained from the data generated by the instrument.

Ignition Loss at 1000° C.

Ignition loss is determined by the loss in weight of a silica when ignited in a furnace at 1000° C. to constant weight.

pH

This measurement is carried out on a 5 weight percent suspension of the silica in boiled demineralised water (CO₂ free).

Guanidine Compatibility Test

The concentration at which the test is carried out depends upon the nature of the silica. When the silica is a thickening silica it is usually necessary to work at a lower concentration so that the viscosity of the mixture is acceptable and good mixing is ensured.

(a) higher concentration test

A measured quantity (usually 4 g) of silica is dispersed in 16 g of a 1% w/v aqueous solution of chlorhexidine digluconate and this suspension is agitated at 37° C. for 24 hrs. The suspension is then centrifuged at 20,000 rpm for 30 min and the supernatant is filtered through a 0.2 μm Millipore filter. 0.5 ml of the filtered solution is withdrawn and diluted to 100 ml with water in a volumetric flask (“Test Solution”). A reference solution is prepared by the same procedure but without the silica. A 1% w/v aqueous solution of chlorhexidine digluconate is agitated at 37° C. for 24 hrs, then centrifuged at 20,000 rpm for 30 min and the supernatant is filtered through a 0.2 μm Millipore filter. 0.5 ml of the filtered solution is withdrawn and diluted to 100 ml with water in a volumetric flask (“Reference Solution”). The absorbance of the two solutions is then measured at 254 nm by means of a double-beam spectrophotometer. The absorbance of water at 254 nm is measured and subtracted as a background from the measured absorbances of both the ‘Test’ and ‘Reference’ solutions to determine the final absorbance values. The compatibility is determined by comparing the amount of chlorhexidine in the two solutions, using the equation:

$$\% compatibility = \frac{absorbance of test solution}{absorbance of reference solution} \times 100$$

(b) lower concentration test

A measured quantity (usually 4 g) of silica is dispersed in 32 g of a 0.5% w/v aqueous solution of chlorhexidine digluconate and this suspension is agitated at 37° C. for 24 hrs. The suspension is then centrifuged at 20,000 rpm for 30 min and the supernatant is filtered through a 0.2 μm Millipore filter. 1 ml of the filtered solution is withdrawn and diluted to 100 ml with water in a volumetric flask (“Test Solution”). A reference solution is prepared by the same procedure but without the silica. A 0.5% w/v aqueous solution of chlorhexidine digluconate is agitated at 37° C. for 24 hrs, then centrifuged at 20,000 rpm for 30 min and the supernatant is filtered through a 0.2 μm Millipore filter. 1 ml of the filtered solution is withdrawn and diluted to 100 ml with water in a volumetric flask (“Reference Solution”). The absorbance of the two solutions is then measured at 254 nm by means of a double-beam spectrophotometer. The absorbance of water at 254 nm is measured and subtracted as a background from the measured absorbances of both the ‘Test’ and ‘Reference’ solutions to determine the final absorbance values. The compatibility is calculated using the equation given for the higher concentration test, (a), above.

The invention will now be further described in the following non-limiting examples in which the first example illustrates one preferred method of preparing a silica treated with polyether glycol, but the invention is not limited to this particular preparation method.

### EXAMPLES

### Example 1

A heated stirred reaction vessel was used for the silicate/acid reaction. Mixing is an important feature in the reaction of silicate and sulphuric acid. Consequently, fixed specifications, as listed in Chemineer Inc. Chem. Eng., 26 Apr. 1976, pages 102-110 have been used to design the baffled, heated stirred reaction vessel. Whilst the turbine design is optional to the mixing geometry, a 6-bladed 30° pitched bladed unit was chosen for the preparation in order to ensure maximum mixing effectiveness with minimum shear.

The solutions used in the process were as follows:

a) Sodium silicate solution with an SiO₂: Na₂O weight ratio of 3.29 and an SiO₂ content of 16.6% by weight

b) A sulphuric acid solution of specific gravity 1.12 (17.4% by weight solution).

The following procedure was adopted for the preparation of a precipitated silica and its subsequent treatment with a polyether glycol.

0.1425 m³ of water was placed in a 0.300 m³ capacity vessel with 1150 cm³ of sodium silicate solution. This mixture was stirred and heated to 94° C. 0.114 m³ of sodium silicate and 0.042 m³ of sulphuric acid were then simultaneously added over 20 minutes at 94° C. The flow rates of the silicate and acid solutions were uniform throughout the addition period to ensure that a constant pH, in the range from 10 to 11, was maintained in the vessel. The slurry was then adjusted with sulphuric acid over a 10-minute period to the final end-of-batch pH, 4.5.

At this point the final slurry was split. One half of the final slurry was filtered and washed with water to remove excess electrolyte. The residual electrolyte was less than 2% on a dry weight basis. The resulting silica is referred to below as the standard silica thickener. To the second half of the final slurry, 393 g of polyethylene glycol with 6000 average molecular weight (PEG 6000) was added and mixing was effected for a period of 30 minutes at a temperature 60° C. and a pH of 4.5. The treated slurry was then filtered and washed in the same manner as described above. The silica derived from the second part is referred to as a silica thickener of the invention. After washing, each filter cake was flash dried to remove the water rapidly from the silica so that the structure was maintained, and comminuted.

The physical properties of the precipitated silicas produced are listed in Table 1. They are suitable for use as thickeners in dentifrice formulations.

TABLE 1

| | Std. Silica | Silica Thickener |
|---|---|---|
| TEST | Thickener | of the Invention |
| Oil Absorption (cm³/100 g) | 233 | 225 |
| pH | 6.4 | 5.9 |
| Weight Mean Particle Size | 14.9 | 11.0 |
| (μm) (Malvern) | | |
| Moisture loss at 105° C. | 4.5 | 5.1 |
| Ignition Loss at 1000° C. | 8.3 | 9.5 |
| SO₄²⁻ (% by weight) | 0.39 | 0.43 |
| PEG 6000 (% by weight) | 0 | 1.27 |

### Examples 2 to 6

A heated stirred vessel, similar to that described in Example 1, but of 0.075 m³ capacity, was used to carry out the polyether glycol treatment. 0.050 m³ of water was added to the vessel and heated to 60° C. 3.5 kg of the chosen, commercially available silica, identified in Table 2, was added to the water and the resultant slurry pH was adjusted to 4.5 by the addition a portion of 17.5% by weight sulphuric acid solution. An amount of polyethylene glycol solution, as defined in Table 2, was then added to the silica slurry and allowed to mix for 30 minutes at 60° C. The resultant treated silica slurry was then filtered using a filter press, washed with 10 litres of water, and flash dried.

TABLE 2

| Ex- | | | PEG loading | % |
|---|---|---|---|---|
| ample | Silica Product | Silica | (% w/w | Compat- |
| No. | Name | Type | on SiO₂) & M. Wt | ibility |
| 2 | Sorbosil ™ TC15 | Thickener | 5% PEG 400 | 61.9¹ |
| 3 | Sorbosil ™ TC15 | Thickener | 1.5% PEG 4000 | 66.4¹ |
| 4 | Sorbosil ™ TC15 | Thickener | 0.25% PEG 6000 | 60.4¹ |
| 5 | Sorbosil ™ TC15 | Thickener | 10% PEG 6000 | 72.2¹ |
| 6 | Sorbosil ™ AC43 | Abrasive | 0.75% PEG 6000 | 70.0² |

¹Tested using the lower concentration test.
²Tested using the higher concentration test.
Sorbosil ™ TC15 and Sorbosil ™ AC43 are commercially available silicas from Ineos Silicas Ltd, Warrington, Cheshire, WA5 1AB.

The loading of PEG on the silica was confirmed by carbon analysis.

### Examples 7 & 8 (Comparative)

The silicas were taken through exactly the same procedure as in Examples 2 to 6, except that polyether glycol was not added to the silica slurry. The results are shown in Table 3 below.

TABLE 3

| Exam- | | | PEG loading | |
|---|---|---|---|---|
| ple | Silica Product | Silica | (% w/w on | % |
| No. | Name | Type | SiO₂) & M. Wt | Compatibility |
| 7 | Sorbosil ™ TC15 | Thickener | None | 0.6¹ |
| 8 | Sorbosil ™ AC43 | Abrasive | None | 1.8² |

¹Tested using the lower concentration test.
²Tested using the higher concentration test.

### Dentifrice Example 9

The oral composition given below is an example of a formulation of a dentifrice in which the silica product, coated with polyether glycols, as described in this invention, can be satisfactorily used.

| Toothpaste Component | % by weight |
|---|---|
| Glycerin | 18.0 |
| Silica abrasive of the | 16.0 |
| invention (RDA value 85) | |
| Hydroxypropyl methylcellulose | 3.6 |
| Chlorhexidine digluconate | 1.0 |
| Flavour | 1.0 |
| Poloxamer 338 | 2.0 |
| Sodium fluoride | 0.23 |
| Deionised water | q.s. |

### Dentifrice Example 10

The oral composition given below is an example of a formulation of a dentifrice in which the silica products coated with polyether glycols, as described in this invention, can be satisfactorily used. Importantly, this is a formulation for a dentifrice containing a silica thickener. The particularly strong interaction of cationic antibacterial agents with silica thickeners has previously created difficulties in the formulation of satisfactory dentifrices containing silica thickeners and cationic antibacterial agents.

| Toothpaste Component | % by weight |
|---|---|
| Glycerin | 18.0 |
| Silica abrasive of the | 9.0 |
| invention (RDA value 125) | |
| Silica thickener of the invention | 6 |
| Hydroxypropyl methylcellulose | 2.5 |
| Chlorhexidine digluconate | 1.0 |
| Flavour | 1.0 |
| Poloxamer 338 | 2.0 |
| Sodium fluoride | 0.23 |
| Deionised water | q.s. |

1. An oral composition comprising a particulate amorphous silica and a cationic antibacterial agent characterised in that the particles of said amorphous silica have a polyether glycol deposited thereon.

2. An oral composition according to claim 1 characterised in that the amorphous silica has a CTAB surface area in the range 5 to 400 m²g⁻¹.

3. An oral composition according to claim 1 characterised in that the amorphous silica has an oil absorption in the range 40 to 400 cm³/100 g.

4. An oral composition according to claim 1 characterised in that the amorphous silica has a weight mean particle size in the range 3 to 20 μm.

5. An oral composition according to claim 1 characterised in that the amorphous silica is in the form of aggregates or agglomerates having a weight mean particle size in the range 50 to 1000 μm.

6. An oral composition according to claim 1 characterised in that the amorphous silica has a pH value, measured as a 5 percent by weight suspension in demineralised water, in the range 5 to 8.

7. An oral composition according to claim 1 characterised in that there is present on the amorphous silica an amount of water, as measured by ignition loss at 1000° C., in the range 4 to 30 percent by weight of the silica.

8. An oral composition according to claim 1 characterised in that the amorphous silica having a polyether glycol deposited thereon has a compatibility with a cationic antibacterial agent, as measured by compatibility with chlorhexidine, of at least 50 percent.

9. An oral composition according to claim 1 characterised in that the amorphous silica having a polyether glycol deposited thereon has a compatibility with a cationic antibacterial agent, as measured by compatibility with chlorhexidine, which is at least 30 percent higher than the compatibility of the silica before treatment with a polyether glycol.

10. An oral composition according to claim 1 characterised in that the composition contains from 0.1 to 35 percent by weight of the amorphous silica having a polyether glycol deposited thereon.

11. An oral composition according to claim 1 characterised in that the polyether glycol is a polyalkylene glycol.

12. An oral composition according to claim 1 characterised in that an amount of polyether glycol in the range 0.1 to 30 per cent by weight with respect to the amorphous silica is deposited on the amorphous silica.

13. An oral composition according to claim 1 characterised in that the polyether glycol is polyethylene glycol having an average molecular weight in the range 200 to 20,000.

14. An oral composition according to claim 1 characterised in that the cationic antibacterial agent is a quaternary ammonium compound, a pyridinium compound, an isoquinolinium compound, a pyrimidine derivative, a bispyridine derivative or a guanide.

15. An oral composition according to claim 14 characterised in that the cationic antibacterial agent is a bis-biguanide of the formula 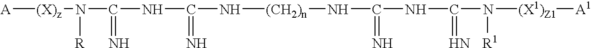
in which A and A¹ each represent (i) a phenyl group optionally substituted by (C₁₋₄) alkyl, (C₁₋₄) alkoxy, nitro, or halogen, (ii) a (C₁₋₁₂) alkyl group, or (iii) a (C₄₋₁₂) alicyclic group, X and X¹ each represent (C₁₋₃) alkylene, R and R¹ each represent hydrogen, (C₁₋₁₂) alkyl, or aryl (C₁₋₆) alkyl, Z and Z1 are each 0 or 1, n is an integer from 2 to 12 and the polymethylene chain (CH₂)_{N} may optionally be interrupted by oxygen, sulphur or an aromatic nucleus, or an orally acceptable acid addition salt of said bis-biguanide.

16. An oral composition according to claim 14 characterised in that the cationic antibacterial agent is selected from the group consisting of benzethonium chloride, octenidine, hexetidine, hexamidine, cetyl pyridinium chloride, chlorhexidine and alexidine.

17. An oral composition according to claim 1 characterised in that the cationic antibacterial agent is present in an amount in the range 0.005 to 10 percent by weight of the oral composition.

18. An oral composition according to claim 1 characterised in that the composition contains a humectant selected from the group consisting of glycerol, sorbitol syrup, polyethylene glycol, polypropylene glycol, lactitol, xylitol and hydrogenated corn syrup in an amount in the range 10 to 85 percent by weight of the oral composition.

19. An oral composition according to claim 1 characterised in that the composition contains a non-ionic, cationic or amphoteric surfactant.

20. An oral composition according to claim 1 characterised in that the composition contains a thickening agent selected from the group consisting of sodium carboxymethyl cellulose, (C₁₋₆) alkylcellulose ethers, hydroxy-(C₁₋₆) alkylcellulose ethers, (C₂₋₆) alkylen oxide modified (C₁₋₆) alkylcellulose ethers, gum tragacanth, polyvinylpyrrolidone, starch, polyacrylates and mixtures thereof.

21. An oral composition according to claim 1 characterised in that the composition contains an ionic fluorine-containing compound in an amount which provides between 100 and 3000 ppm of fluoride to the composition.

22. An oral composition according to claim 1 characterised in that the polyether glycol is a polyalkylene glycol which is deposited on the silica by adding the polyalkylene glycol to an aqueous slurry of a precipitated silica, mixing for 5 to 60 minutes at a temperature in the range 20 to 95° C. and at a pH in the range 2 to 7, filtering the resulting slurry and washing, drying and comminuting the treated silica thus produced.

23. An oral composition according to claim 1 characterised in that the polyether glycol is a polyalkylene glycol which is deposited on the silica by spraying a solution of the polyalkylene glycol onto silica particles in a fluidised bed and the treated silica thus produced is dried and comminuted.
